(19) United States
(12) Patent Application Publication
(10) Pub. No.: US 2022/0233655 A1
(43) Pub. Date: Jul. 28, 2022

(54) IMPROVED DELIVERY OF GENE THERAPY VECTORS TO RETINAL CELLS USING A GLYCOSIDE HYDROLASE ENZYME

(71) Applicant: RESEARCH INSTITUTE AT NATIONWIDE CHILDREN'S HOSPITAL, Columbus, OH (US)

(72) Inventors: Kathrin Christine Meyer, Columbus, OH (US); Shibi Likhite, Columbus, OH (US)

(21) Appl. No.: 17/611,972

(22) PCT Filed: Apr. 15, 2020
(86) PCT No.: PCT/US20/28207

§ 371 Date: Nov. 17, 2021

Related U.S. Application Data
(60) Provisional application No. 62/849,794, filed on May 17, 2019.

(51) Int. Cl. A61K 38/47; A61K 48/00; A61P 27/02

(52) U.S. Cl. CPC A61K 38/47; A61P 27/02; A61K 48/0075; A61K 48/005

(57) ABSTRACT

The present disclosure relates to methods of targeting specific cell types within the retina using optimized gene therapy vectors in combination with a glycoside hydrolase enzyme, such as neuraminidase. In particular, the disclosure provides gene therapy vectors administered with a glycoside hydrolase enzyme to specifically target retinal cells and methods of treating visual impairment, retinal degeneration and vision-related disorders.

This application claims priority to U.S. Provisional Application No. 62/849,794 filed on May 17, 2019 which is incorporated by reference herein in its entirety.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “55603_Seqlisting.txt”, which was created on Apr. 13, 2020 and is 15,459 bytes in size. The subject matter of the Sequence Listing is incorporated herein in its entirety by reference.

### FIELD

The present disclosure relates to methods of targeting specific cell types within the retina using optimized gene therapy vectors in combination with a glycoside hydrolase enzyme. In particular, the disclosure provides gene therapy vectors to specifically target retinal cells and methods of treating visual impairment, retinal degeneration and vision-related disorders.

### BACKGROUND

Ocular administration of gene therapy vectors has many advantages due to the well-defined anatomy of the eye. In particular, the eye's easy accessibility enables rapid and progressive examinations; the relatively enclosed structure and small size of the eye require lower doses of vector for delivery; the blood-retinal barrier prevents the leakage of vectors into systemic circulation, maintaining a relatively immune-privileged environment; and individual or multiple genes primarily or partially involved in particular ocular disorders have been identified.

Ocular administration of gene therapy vectors has shown some promising results. Currently, there are a number of clinical gene therapy trials targeting vision-loss related diseases, and these trials mainly target hereditary retinal disease. For example, clinical trials have investigated Leber congenital amaurosis (LCA), leber's hereditary optic neuropathy and retinitis pigmentosa. To date, AAV vectors, particular AAV2 serotype, have been the most commonly used in ocular gene therapy. See Lee et al., Progress in retinal and eye research 68: 31-53, 2019

Neuronal ceroid lipofuscinoses (NCLs) are a group of severe neurodegenerative disorders, which are collectively referred to as Batten disease. These disorders affect the nervous system and typically cause worsening problems with e.g. movement, vision and thinking ability. The different NCLs are distinguished by their genetic cause. Partial or complete loss of vision often develops in patients who have childhood forms of Batten disease. In particular, in people suffering from Batten disease, lipofuscin accumulates inside cells, including those of the brain and retina. The buildup of lipofuscin damages the photoreceptors in the retina, optic nerve, and area of the brain that processes vision.

Currently, there is a need for improved gene therapy methods that target specific cell types in the retina. Furthermore, there are no therapies that can reverse the symptoms of Batten Disease. Thus, there is a need in the art for treatments for Batten Disease.

### SUMMARY

The disclosure provides compositions comprising an optimized gene therapy vector that targets specific cell types, such as the specific cells in the retina and a glycoside hydrolase enzyme. These optimized gene therapy vectors are useful for delivering a transgene to specific retinal cells using intravitreal delivery in combination with administration of a glycoside hydrolase enzyme. The administration of the glycoside hydrolase enzyme enhanced gene therapy penetration within the retina. The disclosure provides for methods of treating a vision-related disorder comprising administering the optimized gene therapy vectors using intravitreal delivery in combination with administration of a glycoside hydrolase enzyme. Gene therapy methods that target specific cells types in the retina have advantages for treating vision-loss related diseases.

The disclosure provides for compositions comprising a gene therapy vector and a glycoside hydrolase enzyme. For example, the glycoside hydrolase enzyme is neuraminidase, lactase, amylase, chitinase, cellulase, sucrase, maltase, invertase, or lysozyme. In some embodiments, the gene therapy vector is AAV8, AA9 or Anc80.

In exemplary embodiments, the disclosure provides for compositions formulated for local intravenous delivery, sub-retinal delivery, intravitreal delivery, intracerebroventricular or intrathecal delivery. For example, the disclosure provides for compositions in which the gene therapy vector and the glycoside hydrolase enzyme are admixed for administration simultaneously.

In addition, the disclosure provides for kits for delivering a transgene to a cell of a subject comprising a gene therapy vector and a glycoside hydrolase enzyme. For example, the cell is a retinal cell. In exemplary embodiments, the glycoside hydrolase enzyme is neuraminidase, lactase, amylase, chitinase, cellulase, sucrase, maltase, invertase, or lysozyme. In addition, the gene therapy is AAV8, AA9 or Anc80. Optionally, the kit comprises instructions for delivering the gene therapy vector and the glycoside hydrolase enzyme to a subject.

The disclosure provides for methods of delivering a transgene to a cell in a subject comprising administering to the subject i) a gene therapy vector encoding the transgene and ii) a glycoside hydrolase enzyme. For example, the cell is a retinal cell. In some embodiments, the glycoside hydrolase enzyme is neuraminidase, lactase, amylase, chitinase, cellulase, sucrase, maltase, invertase, or lysozyme, and/or the gene therapy vector is AAV8, AAV9 or Anc80. The disclosure provides for methods wherein the gene therapy vector and/or the glycoside hydrolase enzyme is administered to the subject using local intravenous (IV) delivery, sub-retinal delivery, intravitreal delivery, intracerebroventricular delivery, intraparenchymal delivery or intrathecal (cerebrospinal fluid) delivery. For example, the disclosed methods result in delivering the transgene to all retinal cells including but not limited to bipolar cell, rod photoreceptor cell, cone photoreceptor cell, ganglion cell, Mueller glia cell, microglia cell, horizontal cell and/or amacrine cell. Delivery to retinal cells is exemplified herein, however the disclosed methods, compositions and uses may target any cell type in which glycoside hydrolase enzymes clear the receptors on the cell membrane. Other cell types include muscle cells, nerve cells such as astrocytes, neurons, oligodendrocytes and scwann cells.

The disclosure also provides for a composition for delivering a transgene to a cell in a subject, wherein the composition comprises i) a gene therapy vector encoding the transgene and ii) a glycoside hydrolase enzyme. For example, the cell is a retinal cell. In additional embodiments, the disclosure provides for a composition for delivering a transgene to a retinal cell in a subject, wherein the composition comprises a gene therapy vector encoding the transgene, wherein the composition is administered with a second composition comprising a glycoside hydrolase enzyme. For example, the composition is formulated for administering the gene therapy vector using local intravenous delivery, sub-retinal delivery, intravitreous delivery, intracistermal injection, intracerebroventricular delivery, intramuscular delivery or intrathecal injection.

The disclosure also provides for use of a composition for the preparation of a medicament for delivering a transgene to a cell in a subject, wherein the composition comprises i) a gene therapy vector encoding the transgene and ii) a glycoside hydrolase enzyme. For example, the cell is a retinal cell. In some embodiments, the disclosure provides for use of a gene therapy vector encoding a transgene for the preparation of a medicament for delivering a transgene to a retinal cell in a subject, wherein the medicament is administered with a composition comprising a glycoside hydrolase enzyme. In other embodiments, the disclosure provides for use of glycoside hydrolase enzyme for the preparation of a medicament for delivering a transgene to a retinal cell in a subject, wherein the medicament is administered with a composition comprising a gene therapy vector encoding the transgene. For example, the medicament is formulated for administering the gene therapy vector using local intravenous delivery, sub-retinal delivery, intravitreous delivery or intrathecal delivery.

The disclosure also provides for methods of treating visual impairment, retinal degeneration or a vision-related disorder in a subject comprising administering to the subject i) a gene therapy vector encoding a transgene and ii) a glycoside hydrolase enzyme. In some embodiments, the glycoside hydrolase enzyme is neuraminidase, lactase, amylase, chitinase, cellulase, sucrase, maltase, invertase, or lysozyme, and/or the gene therapy vector is AAV8, AAV9 or Anc80. The disclosure provides for methods wherein the gene therapy vector and/or the glycoside hydrolase enzyme is administered using local intravenous (IV) delivery, sub-retinal delivery, intravitreal delivery, intracerebroventricular delivery, intraparenchymal delivery, intramuscular delivery or intrathecal delivery.

The disclosure also provides for compositions for treating visual impairment or a vision-related disorder in a subject, wherein the composition comprises i) a gene therapy vector encoding a transgene and ii) a glycoside hydrolase enzyme. In some embodiments, the disclosure provides for a composition for treating a treating visual impairment or a vision-related disorder in a subject, wherein the composition comprises a glycoside hydrolase enzyme, wherein the composition is administered with a second composition comprising a gene therapy vector encoding the transgene. For example, the composition is formulated for administering the gene therapy vector using local intravenous delivery, sub-retinal delivery, intravitreous delivery, intracerebroventricular delivery, intramuscular delivery or intrathecal delivery.

In additional embodiments, the disclosure provides for use of a composition for the preparation of a medicament for treating visual impairment or a vision-related disorder in a subject, wherein the composition comprises a gene therapy vector encoding a transgene. The disclosure also provides for use of a gene therapy vector encoding a transgene for the preparation of a medicament for treating visual impairment or a vision-related disorder in a subject, wherein the medicament is administered with a composition comprising a glycoside hydrolase enzyme. In addition, the disclosure provides for use of glycoside hydrolase enzyme for the preparation of a medicament for treating a treating visual impairment or a vision-related disorder in a subject, wherein the medicament is administered with a composition comprising a gene therapy vector encoding the transgene. For example, the medicament is formulated for administering the gene therapy vector using local intravenous delivery, sub-retinal delivery, intravitreous delivery, intracerebroventricular delivery, intramuscular delivery or intrathecal delivery.

For example, the vision-related disorder is Batten disease, congenital cataracts, congenital glaucoma, retinal degeneration, optic atrophy, eye malformations. Strabismus, ocular misalignment, glaucoma, wet age-related macular degeneration, dry age-related macular degeneration, retinitis pigmentosa, choroideremia, Leber congenital amaurosis, Leber's hereditary optic neuropathy, early onset retinal dystrophy, achromatopsia, x-linked retinoschisis, Usher Syndrome 1B, neovascular age-related macular degeneration, Stargardt's macular degeneration, diabetic macular degeneration, or diabetic macular edema. In a particular embodiment, the vision-related disorder is a CLN Batten disease such as CLN1 disease, CLN2 disease, CLN3 disease, CLN4 disease, CLN5 disease, CLN6 disease or CLN8 disease.

In any of the disclosed methods, uses or compositions, the transgene is a polynucleotide sequence that encodes a polypeptide of interest or is a nucleic acid that inhibits, interferes or silences expression of a gene of interest, such as a siRNA or miRNA. Exemplary transgenes are polynucleotides that encode RPE65, RPGR, ORF15, CNGA3, CMH, ND4, PDE6B, ChR2, MERTK, hRS1, hMYOJA, hABCA4, CD59, anti-hVEGF antibody, endostatin-angiostatin, sFLT01, or sFLT-1. Additional exemplary transgenes include siRNA against RTP801, siRNA against VEGFR-1, siRNA against VEGF, or siRNA against ADRB2. In one embodiment, the transgene encodes a CLN polypeptide, such as CLN1, CLN2, CLN3, CLN4, CLN5, CLN6 or CLN8.

In any of the disclosed methods, compositions or uses disclosed herein, the gene therapy vector and the glycoside hydrolase enzyme are administered simultaneously, or sequentially. In addition in any of the disclosed methods, compositions or uses, the gene therapy vector and the glycoside hydrolase enzyme are administered using the same mode of delivery or administered for each, or using a different mode of delivery for each. In any of the disclosed methods, uses or compositions, the gene therapy vector and the glycoside hydrolase enzyme are administered as a single administration, for example the gene therapy vector and the glycoside hydrolase enzyme are admixed. Alternatively, the gene therapy vector and the glycoside hydrolase enzyme are administered separately. In some embodiments, the glycoside hydrolase enzyme is administered at least about 30 minutes before administration of the gene therapy vector.

In any of the disclosed methods, uses or compositions, the gene therapy vector is AAV1, AAV2, AAV3, AAV4, AAV5, AAV6, AAV7, AAV8, AAV9, AAVRH10, AAVRH74, AAV11, AAV12, AAV13, AAVTT or Anc80, AAV7m8 and their derivatives.

In any of the disclosed methods, uses or compositions, the gene therapy vector comprises a CMV promoter, the p546, or the CB promoter.

In addition, in any of the disclosed methods, uses or compositions, the gene therapy vector and/or glycoside hydrolase enzyme is administered using intrathecal delivery, and the method further comprises placing the subject in the Trendelenburg position after administering of the gene therapy vector.

In any of the methods, uses and compositions provided the compositions may comprise a non-ionic, low-osmolar contrast agent. For example, the compositions may comprise a non-ionic, low-osmolar contrast agent is selected from the group consisting of iobitridol, iohexol, iomeprol, iopamidol, iopentol, iopromide, ioversol, ioxilan, and combinations thereof.

### BRIEF DESCRIPTION OF THE DRAWINGS

FIG. 1 provides the schematic of the vectors tested in this disclosure.

FIG. 2 demonstrates retinal delivery of the GFP transgene after intravitreal delivery, and demonstrates that administration in combination with neuraminidase enhanced penetration of the transgene.

FIG. 3 provides a schematic detailing the timing of intravitreal injection with and without neuraminidase in 8 week wild-type mice.

FIGS. 4A-4C demonstrate that the addition of neuraminidase significantly increases viral transduction of bipolar cells. The tissues were stained for GFP and Otx which is a bipolar cell specific marker.

### DETAILED DESCRIPTION

Optimization of AAV gene therapy for targeting the eye to treat vision-related disordersrequires specific targeting of different cell types. The disclosure provides experimental data comparing different gene therapy vectors, promoters and routes of administration to determine the optimal gene therapy vector for targeting delivery of a transgene to specific cell types in the retina of mice and non-human primates.

The data focuses on administration of AAV9 and Anc80 vectors, but the disclosure contemplates using any gene therapy vector that comprises a promoter that specifically targets a retinal cell, and these optimized vectors are administered using local intravenous (IV) delivery, sub-retinal delivery, intravitreal delivery, intracerebroventricular delivery, intramuscular delivery, intraparenchymal delivery or intrathecal delivery. For example, the data demonstrated that AAV9 injected directly into the cerebrospinal fluid via intracerebroventricular injection was effective in targeting transgene expression in the bipolar cells of the retina. Thus, intrathecal injections can be used to deliver gene therapy vectors to the eye and specifically for delivering gene therapy vectors to bipolar cells.

### Gene Therapy Vectors

Adeno-associated virus (AAV) is a replication-deficient parvovirus, the single-stranded DNA genome of which is about 4.7 kb in length including two 145 nucleotide inverted terminal repeats (ITRs) and may be used to refer to the virus itself or derivatives thereof. The term covers all subtypes and both naturally occurring and recombinant forms, except where specified otherwise. There are multiple serotypes of AAV. The serotypes of AAV are each associated with a specific clade, the members of which share serologic and functional similarities. Thus, AAVs may also be referred to by the clade. For example, AAV9 sequences are referred to as “clade F” sequences (Gao et al., J. Virol., 78: 6381-6388 (2004). The present disclosure contemplates the use of any sequence within a specific clade, e.g., clade F. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); the AAV-11 genome is provided in Virology, 330(2): 375-383 (2004); portions of the AAV-12 genome are provided in Genbank Accession No. DQ813647; portions of the AAV-13 genome are provided in Genbank Accession No. EU285562. The sequence of the AAV rh.74 genome is provided in see U.S. Pat. No. 9,434,928, incorporated herein by reference. The sequence of the AAV-B 1 genome is provided in Choudhury et al., Mol. Ther., 24(7): 1247-1257 (2016). Anc80 is an AAV vector that is of AAV1, AAV2, AAV8 and AAV9. The sequence of Anc80 is provided in Zinn et al., Cell Reports 12: 1056-1068, 2015, Vandenberghe et al, PCT/US2014/060163, both of which are incorporated by reference herein, in their entirety and GenBank Accession Nos. KT235804-KT235812.

Cis-acting sequences directing viral DNA replication (rep), encapsidation/packaging and host cell chromosome integration are contained within the ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The native AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as a gene cassette containing a promoter, a DNA of interest and a polyadenylation signal. In some instances, the rep and cap proteins are provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

The term “AAV” as used herein refers to the wild type AAV virus or viral particles. The terms “AAV,” “AAV virus,” and “AAV viral particle” are used interchangeably herein. The term “rAAV” refers to a recombinant AAV virus or recombinant infectious, encapsulated viral particles. The terms “rAAV,” “rAAV virus,” and “rAAV viral particle” are used interchangeably herein.

The term “rAAV genome” refers to a polynucleotide sequence that is derived from a native AAV genome that has been modified. In some embodiments, the rAAV genome has been modified to remove the native cap and rep genes. In some embodiments, the rAAV genome comprises the endogenous 5′ and 3′ inverted terminal repeats (ITRs). In some embodiments, the rAAV genome comprises ITRs from an AAV serotype that is different from the AAV serotype from which the AAV genome was derived. In some embodiments, the rAAV genome comprises a transgene of interest flanked on the 5′ and 3′ ends by inverted terminal repeat (ITR). In some embodiments, the rAAV genome comprises a “gene cassette.”

The term “scAAV” refers to a rAAV virus or rAAV viral particle comprising a self-complementary genome. The term “ssAAV” refers to a rAAV virus or rAAV viral particle comprising a single-stranded genome.

The rAAV genomes provided herein, in some embodiments, comprise one or more AAV ITRs flanking the transgene polynucleotide sequence. The transgene polynucleotide sequence is operatively linked to transcriptional control elements (including, but not limited to, promoters, enhancers and/or polyadenylation signal sequences) that are functional in target cells to form a gene cassette. Examples of promoters are the CMV promoter, chicken β actin promoter (CB), and the P546 promoter. Additional promoters are contemplated herein including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1a promoter, the hemoglobin promoter, and the creatine kinase promoter.

Additionally provided herein are a CMV promoter sequence, a CB promoter sequence, a P546 promoter sequence, and promoter sequences at least: 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of the CMV, CB or P546 sequence which exhibit transcription promoting activity.

Other examples of transcription control elements are tissue specific control elements, for example, promoters that allow expression specifically within neurons or specifically within astrocytes. Examples include neuron specific enolase and glial fibrillary acidic protein promoters. Inducible promoters are also contemplated. Non-limiting examples of inducible promoters include, but are not limited to a metallothionine promoter, a glucocorticoid promoter, a progesterone promoter, and a tetracycline-regulated promoter. The gene cassette may also include intron sequences to facilitate processing of a transgene RNA transcript when expressed in mammalian cells. One example of such an intron is the SV40 intron.

“Packaging” refers to a series of intracellular events that result in the assembly and encapsidation of an AAV particle. The term “production” refers to the process of producing the rAAV (the infectious, encapsulated rAAV particles) by the packing cells.

AAV “rep” and “cap” genes refer to polynucleotide sequences encoding replication and encapsidation proteins, respectively, of adeno-associated virus. AAV rep and cap are referred to herein as AAV “packaging genes.”

A “helper virus” for AAV refers to a virus that allows AAV (e.g. wild-type AAV) to be replicated and packaged by a mammalian cell. A variety of such helper viruses for AAV are known in the art, including adenoviruses, herpesviruses and poxviruses such as vaccinia. The adenoviruses may encompass a number of different subgroups, although Adenovirus type 5 of subgroup C is most commonly used. Numerous adenoviruses of human, non-human mammalian and avian origin are known and available from depositories such as the ATCC. Viruses of the herpes family include, for example, herpes simplex viruses (HSV) and Epstein-Barr viruses (EBV), as well as cytomegaloviruses (CMV) and pseudorabies viruses (PRV); which are also available from depositories such as ATCC.

“Helper virus function(s)” refers to function(s) encoded in a helper virus genome which allow AAV replication and packaging (in conjunction with other requirements for replication and packaging described herein). As described herein, “helper virus function” may be provided in a number of ways, including by providing helper virus or providing, for example, polynucleotide sequences encoding the requisite function(s) to a producer cell in trans.

The rAAV genomes provided herein lack AAV rep and cap DNA. AAV DNA in the rAAV genomes (e.g., ITRs) contemplated herein may be from any AAV serotype suitable for deriving a recombinant virus including, but not limited to, AAV serotypes Anc80, AAV-1, AAV-2, AAV-3, AAV-4, AAV-5, AAV-6, AAV-7, AAV-8, AAV-9, AAV-10, AAV-11, AAV-12, AAV-13, AAV rh.74 and AAV-B1. As noted above, the nucleotide sequences of the genomes of various AAV serotypes are known in the art. rAAV with capsid mutations, are also contemplated. See, for example, Marsic et al., Molecular Therapy, 22(11): 1900-1909 (2014). Modified capsids herein are also contemplated and include capsids having various post-translational modifications such as glycosylation and deamidation. Deamidation of asparagine or glutamine side chains resulting in conversion of asparagine residues to aspartic acid or isoaspartic acid residues, and conversion of glutamine to glutamic acid or isoglutamic acid is contemplated in rAAV capsids provided herein. See, for example, Giles et al., Molecular Therapy, 26(12): 2848-2862 (2018). Modified capsids herein are also contemplated to comprise targeting sequences directing the rAAV to the affected tissues and organs requiring treatment.

DNA plasmids provided herein comprise rAAV genomes described herein. The DNA plasmids may be transferred to cells permissible for infection with a helper virus of AAV (e.g., adenovirus, E1-deleted adenovirus or herpesvirus) for assembly of the rAAV genome into infectious viral particles with AAV9 capsid proteins. Techniques to produce rAAV, in which an rAAV genome to be packaged, rep and cap genes, and helper virus functions are provided to a cell are standard in the art. Production of rAAV particles requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs. Production of pseudotyped rAAV is disclosed in, for example, WO 01/83692 which is incorporated by reference herein in its entirety. In various embodiments, AAV capsid proteins may be modified to enhance delivery of the recombinant rAAV. Modifications to capsid proteins are generally known in the art. See, for example, US 2005/0053922 and US 2009/0202490, the disclosures of which are incorporated by reference herein in their entirety.

A method of generating a packaging cell is to create a cell line that stably expresses all the necessary components for rAAV production. For example, a plasmid (or multiple plasmids) comprising a rAAV genome lacking AAV rep and cap genes, AAV rep and cap genes separate from the rAAV genome, and a selectable marker, such as a neomycin resistance gene, may be integrated into the genome of a cell. rAAV genomes may be introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line may then be infected with a helper virus such as adenovirus. The advantages of this method are that the cells are selectable and are suitable for large-scale production of rAAV. Other non-limiting examples of suitable methods employ adenovirus or baculovirus rather than plasmids to introduce rAAV genomes and/or rep and cap genes into packaging cells.

General principles of rAAV particle production are reviewed in, for example, Carter, 1992, Current Opinions in Biotechnology, 1533-539; and Muzyczka, 1992, Curr. Topics in Microbial. and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. Nos. 5,786,211; 5,871,982; and 6,258,595. The foregoing documents are hereby incorporated by reference in their entirety herein, with particular emphasis on those sections of the documents relating to rAAV particle production.

Further provided herein are packaging cells that produce infectious rAAV particles. In one embodiment packaging cells may be stably transformed cancer cells such as HeLa cells, 293 cells and PerC.6 cells (a cognate 293 line). In another embodiment, packaging cells may be cells that are not transformed cancer cells such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

Also provided herein are rAAV (e.g., infectious encapsidated rAAV particles) comprising a rAAV genome of the disclosure. The genomes of the rAAV lack AAV rep and cap DNA, that is, there is no AAV rep or cap DNA between the ITRs of the genomes of the rAAV. The rAAV genome can be a self-complementary (sc) genome. A rAAV with a sc genome is referred to herein as a scAAV. The rAAV genome can be a single-stranded (ss) genome. A rAAV with a single-stranded genome is referred to herein as an ssAAV.

The rAAV may be purified by methods standard in the art such as by column chromatography or cesium chloride gradients. Methods for purifying rAAV from helper virus are known in the art and may include methods disclosed in, for example, Clark et al., Hum. Gene Ther., 10(6): 1031-1039 (1999); Schenpp and Clark, Methods Mol. Med., 69: 427-443 (2002); U.S. Pat. No. 6,566,118 and WO 98/09657.

Compositions comprising rAAV are also provided. Compositions comprise a rAAV encoding a CLN6 polypeptide. Compositions may include two or more rAAV encoding different polypeptides of interest. In some embodiments, the rAAV is scAAV or ssAAV.

Compositions provided herein comprise rAAV and a pharmaceutically acceptable excipient or excipients. Acceptable excipients are nontoxic to recipients and are preferably inert at the dosages and concentrations employed, and include, but are not limited to, buffers such as phosphate [e.g., phosphate-buffered saline (PBS)], citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, copolymers such as poloxamer 188, pluronics (e.g., Pluronic F68) or polyethylene glycol (PEG). Compositions provided herein can comprise a pharmaceutically acceptable aqueous excipient containing a non-ionic, low-osmolar compound such as iobitridol, iohexol, iomeprol, iopamidol, iopentol, iopromide, ioversol, or ioxilan, where the aqueous excipient containing the non-ionic, low-osmolar compound can have one or more of the following characteristics: about 180 mgI/mL, an osmolality by vapor-pressure osmometry of about 322 mOsm/kg water, an osmolarity of about 273 mOsm/L, an absolute viscosity of about 2.3 cp at 20° C. and about 1.5 cp at 37° C., and a specific gravity of about 1.164 at 37° C. Exemplary compositions comprise about 20 to 40% non-ionic, low-osmolar compound or about 25% to about 35% non-ionic, low-osmolar compound. An exemplary composition comprises scAAV or rAAV viral particles formulated in 20 mM Tris (pH8.0), 1 mM MgCl₂, 200 mM NaCl, 0.001% poloxamer 188 and about 25% to about 35% non-ionic, low-osmolar compound. Another exemplary composition comprises scAAV formulated in and 1×PBS and 0.001% Pluronic F68.

Dosages of rAAV to be administered in methods of the disclosure will vary depending, for example, on the particular rAAV, the mode of administration, the time of administration, the treatment goal, the individual, and the cell type(s) being targeted, and may be determined by methods standard in the art. Dosages may be expressed in units of viral genomes (vg). Dosages contemplated herein include about 1×10⁷, 1×10⁸, 1×10⁹, 5×10⁹, 6×10⁹, 7×10⁹, 8×10⁹, 9×10⁹, 1×10¹⁹, 2×10¹⁰, 3×10¹⁰, 4×10¹⁰, 5×10¹⁰, 1×10¹¹, about 1×10¹², about 1×10¹³, about 1.1×10¹³, about 1.2×10¹³, about 1.3×10¹³, about 1.5×10¹³, about 2×10¹³, about 2.5×10¹³, about 3×10¹³, about 3.5×10¹³, about 4×10¹³, about 4.5×10¹³, about 5×10¹³, about 6×10¹³, about 1×10¹⁴, about 2×10¹⁴, about 3×10¹⁴, about 4×10¹⁴, about 5×10¹⁴, about 1×10¹⁵, to about 1×10¹⁶, or more total viral genomes. Dosages of about 1×10⁹ to about 1×10¹⁰, about 5×10⁹ to about 5×10¹⁰, about 1×10¹⁰ to about 1×10¹¹, about 1×10¹¹ to about 1×10¹⁵ vg, about 1×10¹² to about 1×10¹⁵ vg, about 1×10¹² to about 1×10¹⁴ vg, about 1×10¹³ to about 6×10¹⁴ vg, and about 6×10¹³ to about 1.0×10¹⁴ vg are also contemplated. One dose exemplified herein is 6×10¹³ vg. Another dose exemplified herein is 1.5×10¹³ vg.

Methods of transducing target retinal cells with rAAV are provided. The retina cells include bipolar cells, rod photoreceptor cells, cone photoreceptor cell, ganglion cell, Mueller glia cells, microglia cells, horizontal cells or amacrine cells.

The term “transduction” is used to refer to the administration/delivery of the CLN6 polynucleotide to a target cell either in vivo or in vitro, via a replication-deficient rAAV of the disclosure resulting in expression of a functional polypeptide by the recipient cell. Transduction of cells with rAAV of the disclosure results in sustained expression of polypeptide or RNA encoded by the rAAV. The present disclosure thus provides methods of administering/delivering to a subject rAAV encoding a transgene encoded polypeptide by an intrathecal, local IV delivery, intracerebroventricular, intramuscular, sub-retinal injection, intravitreous delivery or intraparenchymal delivery, or any combination thereof. Intrathecal delivery refers to delivery into the space under the arachnoid membrane of the brain or spinal cord. In some embodiments, intrathecal administration is via intracisternal administration.

### Transgenes

The disclosed methods of delivery any transgene of interest to a retinal cell. The transgene is a polynucleotide sequence that encodes a polypeptide of interest or is a nucleic acid that inhibits, interferes or silences expression of a gene of interest, such as a siRNA or miRNA.

Exemplary transgenes are polynucleotides that encode RPE65, RPGR, ORF15, CNGA3, CMH, ND4, PDE6B, ChR2, MERTK, hRS1, hMYOJA, hABCA4, CD59, anti-hVEGF antibody, endostatin-angiostatin, sFLT01, or sFLT-1. In one embodiment, the transgene encodes a CLN polypeptide, such as CLN1, CLN2, CLN3, CLN4, CLN5, CLN6 or CLN8. Additional exemplary transgenes include siRNA against RTP801, siRNA against VEGFR-1, siRNA against VEGF, or siRNA against ADRB2.

miRNA that are expressed in the retina are contemplated as transgenes to include in the disclosed optimized gene therapy vectors. Examples of miRNA are provided in Karali et al., Nucleic Acids Res. 2016 Feb. 29; 44(4): 1525-1540, which is incorporated by reference herein.

rAAV genomes provided herein may comprise a polynucleotide encoding a transgene comprising a polynucleotide sequence encoding any one of RPE65, RPGR, ORF15, CNGA3, CMH, ND4, PDE6B, ChR2, MERTK, hRS1, hMYOJA, hABCA4, CD59, PEDF, endostatin-angiostatin genes, sFLT-1, gene encoding an anti-hVEGF antibody. For example, the polypeptide encoded by the transgene include polypeptides comprising an amino acid sequence that is at least: 90%, 91%, 92%, 93%, 94%, 95%, 96%, 97%, 98%, or 99% identical to the amino acid sequence encoded by the transgene sequence.

rAAV genomes provided herein comprise a polynucleotide encoding a CLN polypeptide, such as CLN1, CLN2, CLN3, CLN4, CLN5, CLN6 and CLN8. The polypeptide include polypeptides comprising an amino acid sequence that is at least: 90%, 91%, 92%, 93%, 94%, 95%, 96%, 97%, 98%, or 99% identical to a CLN polypeptide amino acid sequence, and which encodes a polypeptide with CLN activity (e.g., at least one of increasing clearance of lysosomal auto fluorescent storage material, reducing lysosomal accumulation of ATP synthase subunit C, and reducing activation of astrocytes and microglia in a patient when treated as compared to, e.g. the patient prior to treatment).

rAAV genomes provided herein, in some cases, comprise a polynucleotide encoding a CLN polypeptide or a polynucleotide at least: 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence that encodes a polypeptide with CLN activity (e.g., at least one of increasing clearance of lysosomal auto fluorescent storage material, reducing lysosomal accumulation of ATP synthase subunit C, and reducing activation of astrocytes and microglia in a patient when treated as compared to, e.g. the patient prior to treatment).

rAAV genomes provided herein, in some embodiments, comprise a transgene comprising a polynucleotide sequence that encodes a polypeptide with a desired activity and that hybridizes under stringent conditions to any one of nucleic acid sequence of a known transgene of interest, or the complement thereof. In other embodiments, rAAV genomes provided herein comprise a polynucleotide sequence that encodes a polypeptide with CLN activity and that hybridizes under stringent conditions to any one of nucleic acid sequences encoding a CLN polypeptide, or the complement thereof.

The following outlines the disease characteristics of each Batten Disease subtype with emphasis on visual components. The data included in the “Primary Affected Retinal Cell” column was determined based on single cell RNA data compiled from mouse retina. Investigation of this data is still ongoing.

| Batten | Affected | Proposed Gene | Disease | Vision Loss | Primary Affected Retinal |
|---|---|---|---|---|---|
| Disease | Gene | Function | Onset | Onset | Cell |
| CLN1 | PPT1 | Lysosomal enzyme | 6-24 | 2 years | Muller Glia, Ganglion, |
| | | | months | | Horizontal, Amacrine |
| CLN2 | TPP1 | Lysosomal Enzyme | 2-4 years | 4-6 years | Mueller Glia |
| CLN3 | CLN3 | Transmembrane | 4-8 years | Initial Symptom | Mueller Glia |
| | | Protein | | | |
| CLN4 | DNAJC5 | Cytoplasmic Protein | ~30 years | Uncommon | Broad [high] expression in |
| | | | | | all cell types |
| CLN5 | CLN5 | Soluble Lysosomal | 4.5-7 | 5-11 years | Mueller Glia |
| | | Protein | years | | |
| CLN6 | CLN6 | Transmembrane | 18 mos-8 | Initial Symptom | Cone and Rod Bipolars |
| | | Protein | years | | |
| CLN7 | MFSD8 | Transmembrane | 2-7 years | Initial Symptom | Broad [low] expression in |
| | | Protein | | | all cell types |
| CLN8 | CLN8 | Transmembrane | 2-6 years | 2-10 years | Mueller Glia |
| | | Protein | | | |
| CLN9 | Unknown | Unknown | 4-10 years | Initial Symptom | Unknown |
| CLN10 | CTSD | Lysosomal Enzyme | At birth | Not Characterized | Broad [high] expression in |
| | | | | due to early death | all cell types |
| CLN11 | GRN | Secretory Pathway | 15-50 | Initial symptom | Mueller glia, Microglia |
| | | Protein | years | | |
| CLN12 | ATP13A2 | Transmembrane | ~8 years | None | Broad [med-high] |
| | | Protein | | | expression in all cell types |
| CLN13 | CTSF | Lysosomal Enzyme | ~30 years | No vision loss | Broad [high] expression in |
| | | | | | all cell types |
| CLN14 | KCTD7 | Cytoplasmic Protein | 8-24 | Varies; ~4 years | Broad [low] expression in |
| | | | months | | all cell types |

The term “stringent” is used to refer to conditions that are commonly understood in the art as stringent. Hybridization stringency is principally determined by temperature, ionic strength, and the concentration of denaturing agents such as formamide. Examples of stringent conditions for hybridization and washing include but are not limited to 0.015 M sodium chloride, 0.0015 M sodium citrate at 65-68° C. or 0.015 M sodium chloride, 0.0015M sodium citrate, and 50% formamide at 42° C. See, for example, Sambrook et al., Molecular Cloning: A Laboratory Manual, 2nd Ed., Cold Spring Harbor Laboratory, (Cold Spring Harbor, N.Y. 1989).

### Methods of Administration

Intrathecal administration is exemplified herein. Intrathecal delivery refers to delivery into the space under the arachnoid membrane of the brain or spinal cord. In some embodiments, intrathecal administration is via intracisternal injection or intralumbar injection. These methods include transducing target cells with one or more rAAV described herein. In some embodiments, the rAAV viral particle comprising a transgene is administered or delivered the eye, brain and/or spinal cord of a patient. In some embodiments, the polynucleotide is delivered to brain. Areas of the brain contemplated for delivery include, but are not limited to, the motor cortex, visual cortex, cerebellum and the brain stem. In some embodiments, the polynucleotide is delivered to the spinal cord. In some embodiments, the polynucleotide is delivered to a lower motor neuron. The polynucleotide may be delivered a retinal cell such as a bipolar cell, rod photoreceptor cell, cone photoreceptor cell, ganglion cell, Mueller glia cell, microglia cell, horizontal cell or amacrine cell.

In some embodiments of methods provided herein, the patient is held in the Trendelenburg position (head down position) after administration of the rAAV (e.g., for about 5, about 10, about 15 or about 20 minutes). For example, the patient may be tilted in the head down position at about 1 degree to about 30 degrees, about 15 to about 30 degrees, about 30 to about 60 degrees, about 60 to about 90 degrees, or about 90 to about 180 degrees).

For sub-retinal administration, a small scleral incision is made, at or posterior to the equator of the eye with a needle, e.g. 30 G needle. Virus or vehicle is delivered sub-retinally via the incision, for example, a fine glass pipette attached by tubing to a Hamilton syringe or via 30 G needle and Hamilton syringe. For example, sub-retinal administration is carried out by a clinically trained surgeon using methods known in the art.

For intracerebroventicular injections, a needle is inserted into the skull and the liquid is injected into the ventricles containing cerebrospinal fluid. For example, intracerebroventicular injections are carried out by a clinically trained surgeon using methods known in the art.

In any of the methods of administration, compositions may comprise a non-ionic, low-osmolar contrast agent. For example, the compositions may comprise a non-ionic, low-osmolar contrast agent is selected from the group consisting of iobitridol, iohexol, iomeprol, iopamidol, iopentol, iopromide, ioversol, ioxilan, and combinations thereof.

The methods provided herein comprise the step of administering an effective dose, or effective multiple doses, of a composition comprising a rAAV provided herein to a subject (e.g., an animal including, but not limited to, a human patient) in need thereof. If the dose is administered prior to development of the symptoms of the vision-related disorder, the administration is prophylactic. If the dose is administered after the development of symptoms of the vision-related disorder, the administration is therapeutic. An effective dose is a dose that alleviates (eliminates or reduces) at least one symptom associated with the vision-related disorder, that slows or prevents progression of the disorder, that diminishes the extent of disorder, that results in remission (partial or total) of disorder, and/or that prolongs survival and/or vision. In comparison to the subject before treatment or in comparison to an untreated subject, methods provided herein result in stabilization, reduced progression of vision loss or retinal degeneration, or improvement in vision or macular degeneration.

When the vision-related disorder is CLN Batten disease, comparison to the subject before treatment or in comparison to an untreated subject, methods provided herein result in stabilization, reduced progression, or improvement in one or more of the scales that are used to evaluate progression and/or improvement in CLN Batten-disease, e.g. the Unified Batten Disease Rating System (UBDRS) or the Hamburg Motor and Language Scale. The UBDRS assessment scales (as described in Marshall et al., Neurology. 2005 65(2):275-279) [including the UBDRS physical one or more of the scales that are used to evaluate progression and/or improvement in CLN Batten-disease, e.g. the Unified Batten Disease Rating System (UBDRS) or the Hamburg Motor and Language Scale. The UBDRS assessment scales (as described in Marshall et al., Neurology. 2005 65(2):275-279) [including the UBDRS physical assessment scale, the UBDRS seizure assessment scale, the UBDRS behavioral assessment scale, the UBDRS capability assessment scale, the UBDRS sequence of symptom onset, and the UBDRS Clinical Global Impressions (CGI)]; the Pediatric Quality of Life Scale (PEDSQOL) scale, motor function, language function, cognitive function, and survival. In comparison to the subject before treatment or in comparison to an untreated subject, methods provided herein may result in one or more of the following: reduced or slowed lysosomal accumulation of autofluorescent storage material, reduced or slowed lysosomal accumulation of ATP Synthase Subunit C, reduced or slowed glial activation (astrocytes and/or microglia) activation; reduced or slowed astrocytosis, and showed a reduction or delay in brain volume loss measured by MRI.

### Glycoside Hydrolase Enzyme

The disclosure provides for administering an optimized gene therapy in vector in combination with a glycoside hydrolase enzyme, such as neuraminidase, lactase, amylase, chitinase, cellulase, sucrase, maltase, invertase, or lysozyme. Exo-glucosidases remove terminal sialic acids from glycan chains. These sialic acids are found on the outermost end of the glycan chains of all cell types and on most secreted proteins. Glycoside hydrolase enzymes are known to clear cell membrane residues, it is thought that this clearance allows for greater viral receptor targeting and cell entry. For example, neuramindase treatment removes N-linked galactosyl residues, thereby enhancing penetration of AAV that use N-linked galactose residues as receptors for entry into the cell, e.g. AAV9 (see Shen et al. J. Biol. Chem. 286:13532-13540, 2011). Delivery to retinal cells is exemplified herein, however the disclosed methods, compositions and uses may target any cell type in which glycoside hydrolase enzymes clear the receptors on the cell membrane.

Combination therapies comprising the optimized gene therapy vectors are also provided. The terms “combination therapy” and “combination treatment” refer to administration of a disclosed optimized gene therapy vector with an agent that improves targeting to a retina cell such as an enzyme. In some embodiments, the methods comprise administering a glycoside hydrolase enzyme to the subject in combination with administration of the gene therapy vector. For example, the glycoside hydrolase enzyme is neuraminidase, lactase, amylase, chitinase, cellulase, sucrase, maltase, invertase, or lysozyme. The agents can be administered simultaneously or sequentially with the vector by the any of the routes of administration described herein.

Combination as used herein includes either simultaneous treatment or sequential treatment. For example, the gene therapy vector and the glycoside hydrolase enzyme are administered simultaneously using the same mode of administration, or the gene therapy vector and the glycoside hydrolase enzyme are administered simultaneously each using a different mode of administration. In an additional example, the gene therapy vector and the glycoside hydrolase enzyme are administered sequentially using the same mode of administration, or the gene therapy vector and the glycoside hydrolase enzyme are administered sequentially each using a different mode of administration. Combinations of methods described herein with standard medical treatments are specifically contemplated.

In some embodiments, the optimized gene therapy vector is administered simultaneously with a glycoside hydrolase enzyme, such as neuraminidase. In other embodiments, the optimized gene therapy vector is administered immediately before or immediately after a glycoside hydrolase enzyme, such as neuraminidase. In other embodiments, the optimized gene therapy vector is administered within about 15 minutes, within about 20 minutes, within about 25 minutes, within about 30 minutes, within about 45 minutes, within 1 hour, within 2 hours, within 3 hours, within 4 hours, within 5 hours, within 6 hours, within 7 hours, within 8 hours, within 9 hours, within 12 hours, within 24 hours, within 36 hours, or within 48 hours of administration of a glycoside hydrolase enzyme, such as neuraminidase. In some embodiments, the glycoside hydrolase enzyme is administered before administration of the optimized gene therapy vector or after administration of the optimized gene therapy vector.

In some embodiments, the optimized gene therapy vector and the glycoside hydrolase enzyme, such as neuraminidase, is administered in a single intravitreal injection. However, the disclosure also provides for administering the combination of the optimized gene therapy vector an the glycoside hydrolase enzyme, such as neuraminidase, in separate intravitreal injections. For example, the glycoside hydrolase enzyme, e.g. neuramidase, is administered about 30 minutes before the administration of the optimized gene therapy vector. In addition, the optimized gene therapy vector an the glycoside hydrolase enzyme may be administered using different modes of administration.

### EXAMPLES

While the following examples describe specific embodiments, it is understood that variations and modifications will occur to those skilled in the art. Accordingly, only such limitations as appear in the claims should be placed on the invention.

### Example 1

### Production of scAAV9.GFP and Anc80.GFP

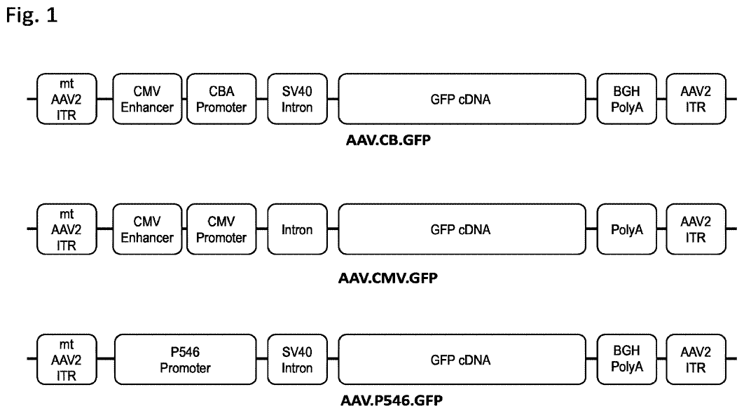

A human GFP cDNA clone was obtained from Origene, Rockville, Md. GFP cDNA was further subcloned into a self complementary AAV9 genome or an Anc80 genome under the hybrid chicken β-Actin promoter (CB), the CMV enhancer-promoter, or the P546 promoter and tested in vitro and in vivo. A schematic of the plasmid constructs showing the GFP cDNA inserted between AAV2 ITRs is provided in FIG. 1. The plasmid construct also included one or more of the CB promoter, an intron such as the simian virus 40 (SV40) chimeric intron and a Bovine Growth Hormone (BGH) polyadenylation signal (BGH PolyA). The constructs in FIG. 1 were packaged into either AAV9 genome or the Anc80 genome (referred to collectively as “AAV”).

### Example 2

### Intavitreal Injection of scAAV9.CB.GFP in Combination with Neuraminidase

The mouse was is anesthetized for the intravitreal injection as described above. A small incision was made between the lumbus and sclera with 30 G needle. Virus or vehicle is delivered into vitreous space via the incision using a fine glass pipette attached by tubing to a Hamilton syringe or via 30 G needle and Hamilton syringe. Before and after the injection, ophthaine and vetropolycin are applied topically, mice are allowed to recover via standard of care (heated cage for recovery, food on the bottom of cage, long sipper tube) and monitored until stable

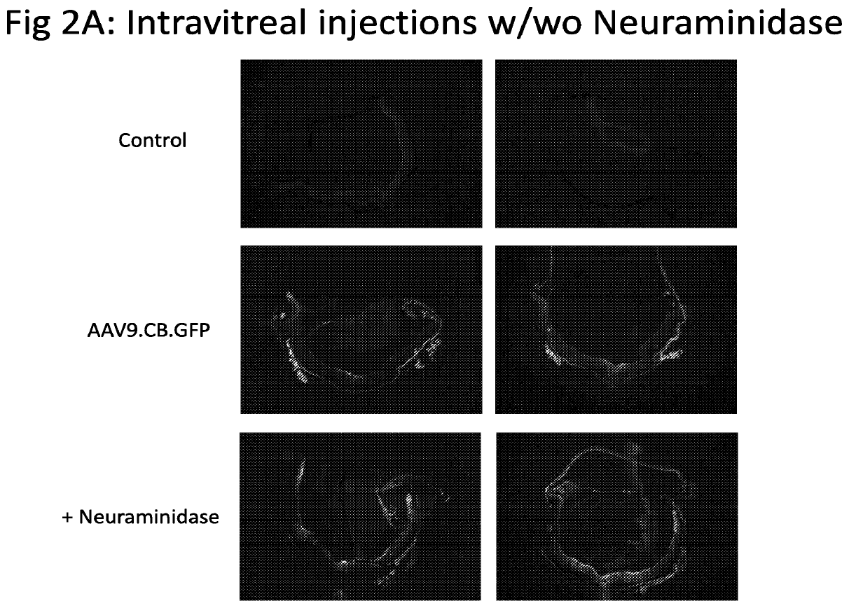

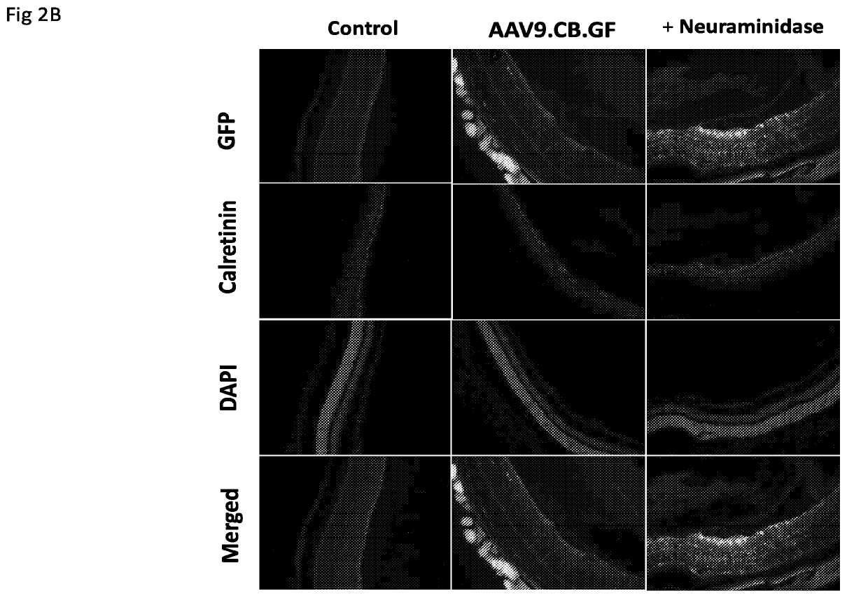

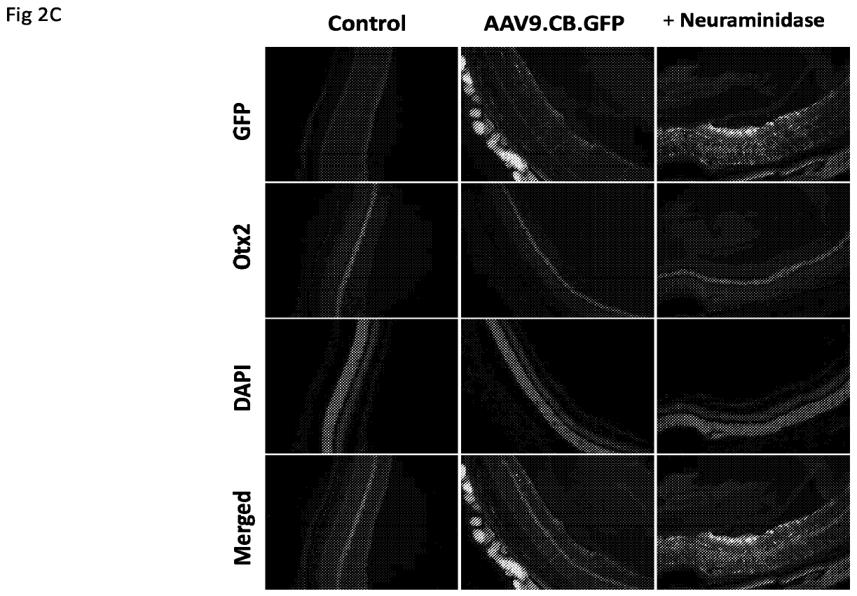

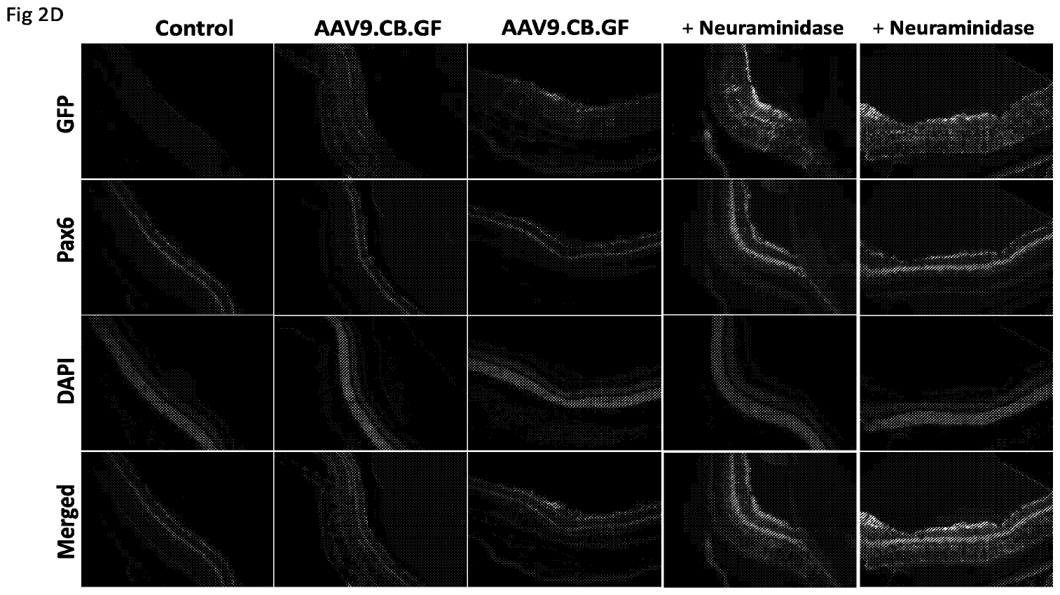

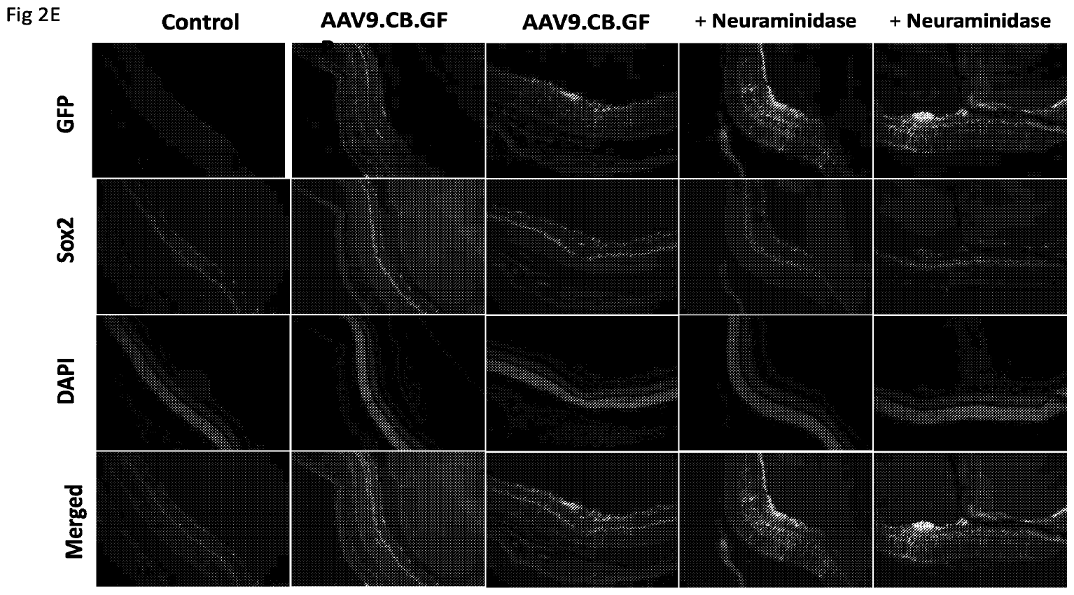

scAAV9.CB.GFP was administered to mice (1-5 months old) via one intravitreal injection and expression was monitored at various time points over a course of two months. The AAV and Anc80 were administered at a dose ranging from 9×10⁹ and 3.2×10¹⁰ vg which was diluted in PBS or straight rAAVinjected. In addition, the rAAV was administered simultaneously with neuraminidase or without neuraminidase within a single intravitreal injection. For the simultaneous administration, the rAAV was admixed with neuraminidase immediately prior to injection and applied as a single solution. As shown in FIG. 2A, intravitreal injections results in expression of the GFP in the retina. Furthermore, administration of the scAAV9.CB.GFP in combination with neuraminidase enhanced penetration of the transgene into the outer layers of the retina. A key of the retinal staining markers is set out below:

| Retinal Cell Type | Retinal Layer | Antibody |
|---|---|---|
| Bipolar Cells (All) | Inner Nuclear Layer (INL) | Otx2 |
| Bipolar Cells (Rod) | Inner Nuclear Layer | PKCα |
| Mueller Glia | All w/nuclei in the INL | Sox2 |
| Photoreceptors (Rod) | Outer Nuclear Layer | Rhodopsin |
| Amacrine | Inner Nuclear Layer | Pax6 |
| Horizontal | Inner Nuclear Layer | Calretinin |
| Microglia | Inner Nuclear Layer | Iba1 |

Calretinin is a makers for horizontal cells (red stain) and stains the inner nuclear layer of the retina. As shown in FIG. 2B, intrvitreal injection of and AAV9.CB.GFP, delivered the transgene to the horizontal cells, and neuraminidase enhanced penetration of the transgene into the inner nuclear layer of the retina.

Otx2 is a nuclear marker for all bipolar cells (red stain) and stains the inner nuclear layer of the retina. As shown in FIG. 2C, intravitreal injection of AAV9.CB.GFP, delivered the transgene to bipolar cells, and neuraminidase enhanced penetration of the transgene into the inner nuclear layer of the retina.

Pax6 is a is a maker for amacrine cells (red stain) and stains the inner nuclear layer of the retina. As shown in FIG. 2D, intravitreal injection of AAV9.CB.GFP delivered the transgene to the amacrine cells, and neuraminidase enhanced penetration of the transgene into the inner nuclear layer of the retina.

Sox2 is a is a maker for Mueller glia cells (red stain) and stains all the nuclei in the inner layer of the retina. As shown in FIG. 2E, intravitreal injection of AAV9.CB.GFP delivered the transgene to the Mueller glia cells, and neuraminidase enhanced penetration of the transgene into the inner nuclear layer of the retina.

### Example 3

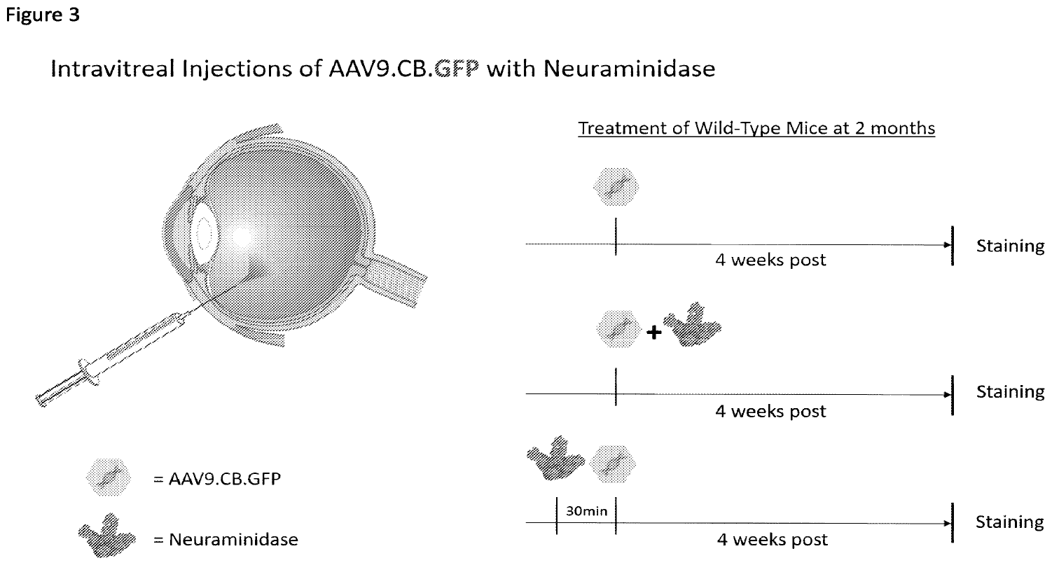

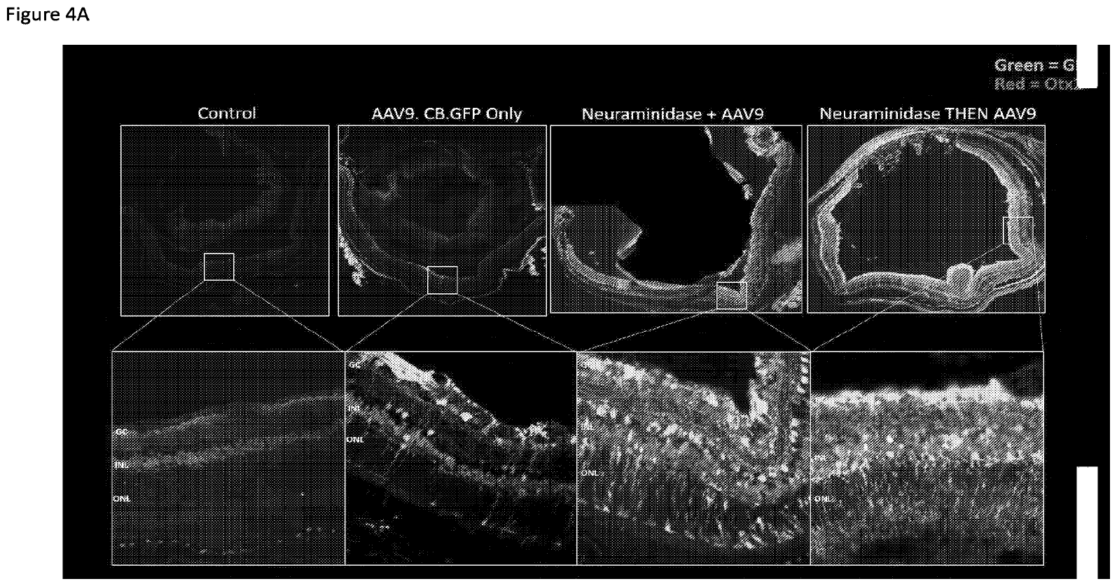

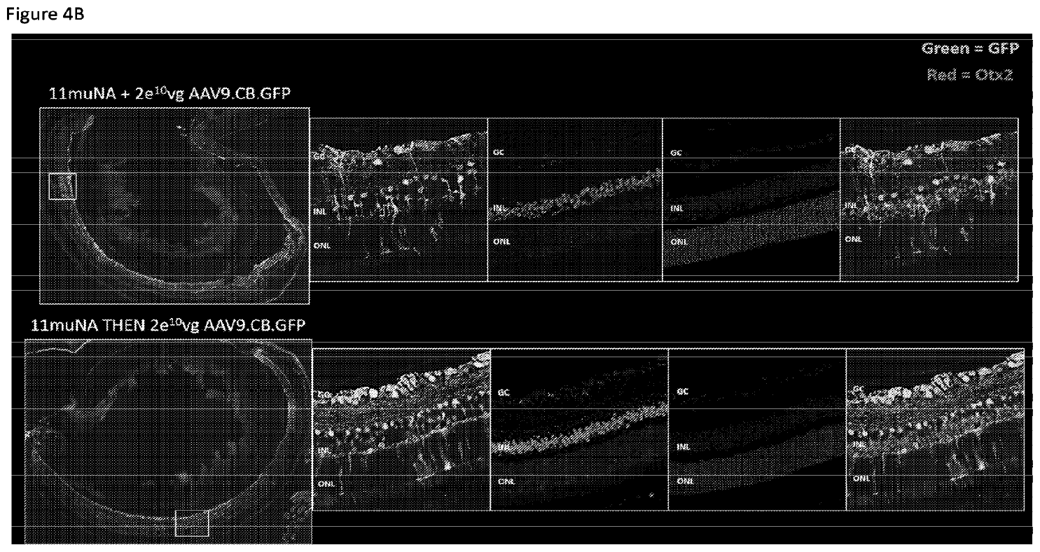

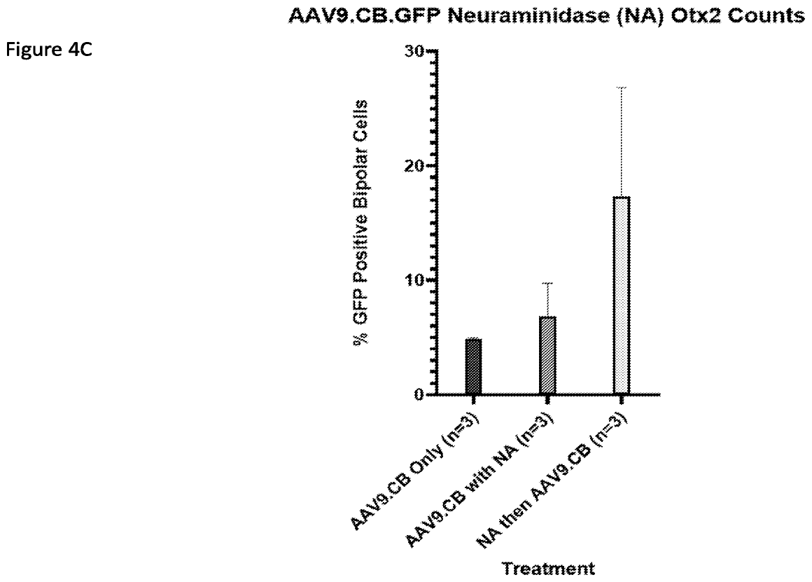

To further investigate the effect of neuraminidase on transduction of 8 week old wild type mice received intravitreal injections of AAV9.CB.GFP with or without neuraminidase as depicted in FIG. 3. The ˜2×10¹⁰ vg of AAV9.CB.GFP was injected in the mice as described in detail in Example 2. The retinal tissue in FIG. 4A and FIG. 4B was stained for transgene GFP and the bipolar cell specific marker Otx2. The addition of neuraminidase either before or after intravitreal injection of the AAV vector significantly increased viral transduction of the biopolar cells (See FIG. 4C).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

All documents referred to in this application are hereby incorporated by reference in their entirety.

### REFERENCES

Arsov, T., Smith, K. R., Damiano, J., Franceschetti, S., Canafoglia, L., Bromhead, C. J., . . . Berkovic, S. F. (2011). Kufs disease, the major adult form of neuronal ceroid lipofuscinosis, caused by mutations in CLN6. American Journal of Human Genetics, 88(5), 566-573. https://doi.org/10.1016/j.ajhg.2011.04.004
Bras, J., Verloes, A., Schneider, S. A., Mole, S. E., & Guerreiro, R. J. (2012). Mutation of the parkinsonism gene ATP13A2 causes neuronal ceroid-lipofuscinosis. Human Molecular Genetics, 21(12), 2646-2650. https://doi.org/10.1093/hmg/dds089
Canafoglia, L., Morbin, M., Scaioli, V., Pareyson, D., D'Incerti, L., Fugnanesi, V., . . . Franceschetti, S. (2014). Recurrent generalized seizures, visual loss, and palinopsia as phenotypic features of neuronal ceroid lipofuscinosis due to progranulin gene mutation. Epilepsia, 55(6), e56-e59. https://doi.org/10.1111/epi.12632
Cannelli, N., Cassandrini, D., Bertini, E., Striano, P., Fusco, L., Gaggero, R., . . . Santorelli, F. M. (2006). Novel mutations in CLN8 in Italian variant late infantile neuronal ceroid lipofuscinosis: another genetic hit in the Mediterranean. Neurogenetics, 7(2), 111-117. https://doi.org/10.1007/s10048-005-0024-y
Estrada-Cuzcano, A., Martin, S., Chamova, T., Synofzik, M., Timmann, D., Holemans, T., . . . Schüle, R. (2017). Loss-of-function mutations in the ATP13A2/PARKS gene cause complicated hereditary spastic paraplegia (SPG78). Brain: A Journal of Neurology, 140(2), 287-305. https://doi.org/10.1093/brain/aww307
Kniffin, C. (2016). OMIM Clinical Synopsis—%609055-CEROID LIPOFUSCINOSIS, NEURONAL, 9; CLN9. Retrieved Mar. 19, 2019, from http://omim.org/clinicalSynopsis/609055
Moen, M. N., Fjer, R., Hamdani, E. H., Laerdahl, J. K., Menchini, R. J., Vigeland, M. D., . . . Chaudhry, F. A. (2016). Pathogenic variants in KCTD7 perturb neuronal K⁺fluxes and glutamine transport. Brain, 139(12), 3109-3120. https://doi.org/10.1093/brain/aww244
Mole, S. E., & Williams, R. E. (1993). Neuronal Ceroid-Lipofuscinoses. GeneReviews®. University of Washington, Seattle. Retrieved from http://www.ncbi.nlm.nih.gov/pubmed/20301601
Nosková, L., Stránecký, V., Hartmannová, H., Přistoupilová, A., Barešová, V., Ivánek, R., . . . Kmoch, S. (2011). Mutations in DNAJCS, encoding cysteine-string protein alpha, cause autosomal-dominant adult-onset neuronal ceroid lipofuscinosis. American Journal of Human Genetics, 89(2), 241-252. https://doi.org/10.1016/j.ajhg.2011.07.003
Roosing, S., van den Born, L. I., Sangermano, R., Banfi, S., Koenekoop, R. K., Zonneveld-Vrieling, M. N., . . . Hoyng, C. B. (2015). Mutations in MFSD8, Encoding a Lysosomal Membrane Protein, Are Associated with Nonsyndromic Autosomal Recessive Macular Dystrophy. Ophthalmology, 122(1), 170-179. https://doi.org/10.1016/j.ophtha.2014 0.07 0.040
Staropoli, J. F., Karaa, A., Lim, E. T., Kirby, A., Elbalalesy, N., Romansky, S. G., . . . Cotman, S. L. (2012). A homozygous mutation in KCTD7 links neuronal ceroid lipofuscinosis to the ubiquitin-proteasome system. American Journal of Human Genetics, 91(1), 202-208. https://doi.org/10.1016/j.ajhg.2012.05.023
Weizmann Institute of Science. (2019). Ceroid Lipofuscinosis, Neuronal, 10 disease: Malacards—Research Articles, Drugs, Genes, Clinical Trials. Retrieved Mar. 19, 2019, from https://www.malacards.org/card/ceroid_lipofuscinosis_neuronal_10_2
Xin, W., Mullen, T. E., Kiely, R., Min, J., Feng, X., Cao, Y., . . . Sims, K. (2010). CLN5 mutations are frequent in juvenile and late-onset non-Finnish patients with NCL. Neurology, 74(7), 565-571. https://doi.org/10.1212/WNL.0b013e3181cff70d

```
CLN3 nucleotide sequence
(SEQ ID NO: 1)
atgggaggct gtgcaggctc gcggcggcgc ttttcggatt ccgaggggga ggagaccgtc 60
ccggagcccc ggctccctct gttggaccat cagggcgcgc attggaagaa cgcggtgggc 120
ttctggctgc tgggcctttg caacaacttc tcttatgtgg tgatgctgag tgccgcccac 180
gacatcctta gccacaagag gacatcggga aaccagagcc atgtggaccc aggcccaacg 240
ccgatccccc acaacagctc atcacgattt gactgcaact ctgtctctac ggctgctgtg 300
ctcctggcgg acatcctccc cacactegtc atcaaattgt tggctcctct tggccttcac 360
ctgctgccct acagcccccg ggttctcgtc agtgggattt gtgctgctgg aagcttcgtc 420
ctggttgcct tttctcattc tgtggggacc agcctgtgtg gtgtggtctt cgctagcatc 480
tcatcaggcc ttggggaggt caccttcctc tccctcactg ccttctaccc cagggccgtg 540
atctcctggt ggtcctcagg gactggggga gctgggctgc tgggggccct gtcctacctg 600
ggcctcaccc aggccggcct ctcccctcag cagaccctgc tgtccatgct gggtatccct 660
gccctgctgc tggccagcta tttcttgttg ctcacatctc ctgaggccca ggaccctgga 720
ggggaagaag aagcagagag cgcagcccgg cagcccctca taagaaccga ggccccggag 780
tcgaagccag gctccagctc cagcctctcc cttcgggaaa ggtggacagt gttcaagggt 840
ctgctgtggt acattgttcc cttggtcgta gtttactttg ccgagtattt cattaaccag 900
ggactttttg aactcctctt tttctggaac acttccctga gtcacgctca gcaataccgc 960
tggtaccaga tgctgtacca ggctggcgtc tttgcctccc gctcttctct ccgctgctgt 1020
cgcatccgtt tcacctgggc cctggccctg ctgcagtgcc tcaacctggt gttcctgctg 1080
gcagacgtgt ggttcggctt tctgccaagc atctacctcg tcttcctgat cattctgtat 1140
gaggggctcc tgggaggcgc agcctacgtg aacaccttcc acaacatcgc cctggagacc 1200
agtgatgagc accgggagtt tgcaatggcg gccacctgca tctctgacac actggggatc 1260
tccctgtegg ggctcctggc tttgcctctg catgacttcc tctgccagct ctcctga 1317
CLN3 amino acid sequence
(SEQ ID NO: 2)
Met Gly Gly Cys Ala Gly Ser Arg Arg Arg Phe Ser Asp Ser Glu Gly
1 5 10 15
Glu Glu Thr Val Pro Glu Pro Arg Leu Pro Leu Leu Asp His Gln Gly
              20 25 30
Ala His Trp Lys Asn Ala Val Gly Phe Trp Leu Leu Gly Leu Cys Asn
        35 40 45
Asn Phe Ser Tyr Val Val Met Leu Ser Ala Ala His Asp Ile Leu Ser
    50 55 60
His Lys Arg Thr Ser Gly Asn Gln Ser His Val Asp Pro Gly Pro Thr
65 70 75 80
Pro Ile Pro His Asn Ser Ser Ser Arg Phe Asp Cys Asn Ser Val Ser
                        85 90 95
Thr Ala Ala Val Leu Leu Ala Asp Ile Leu Pro Thr Leu Val Ile Lys
                100 105 110
Leu Leu Ala Pro Leu Gly Leu His Leu Leu Pro Tyr Ser Pro Arg Val
        115 120 125
Leu Val Ser Gly Ile Cys Ala Ala Gly Ser Phe Val Leu Val Ala Phe
    130 135 140
Ser His Ser Val Gly Thr Ser Leu Cys Gly Val Val Phe Ala Ser Ile
145 150 155 160
Ser Ser Gly Leu Gly Glu Val Thr Phe Leu Ser Leu Thr Ala Phe Tyr
                        165 170 175
Pro Arg Ala Val Ile Ser Trp Trp Ser Ser Gly Thr Gly Gly Ala Gly
                180 185 190
Leu Leu Gly Ala Leu Ser Tyr Leu Gly Leu Thr Gln Ala Gly Leu Ser
        195 200 205
Pro Gln Gln Thr Leu Leu Ser Met Leu Gly Ile Pro Ala Leu Leu Leu
    210 215 220
Ala Ser Tyr Phe Leu Leu Leu Thr Ser Pro Glu Ala Gln Asp Pro Gly
225 230 235 240
Gly Glu Glu Glu Ala Glu Ser Ala Ala Arg Gln Pro Leu Ile Arg Thr
                  245 250 255
Glu Ala Pro Glu Ser Lys Pro Gly Ser Ser Ser Ser Leu Ser Leu Arg
            260 265 270
Glu Arg Trp Thr Val Phe Lys Gly Leu Leu Trp Tyr Ile Val Pro Leu
      275 280 285
Val Val Val Tyr Phe Ala Glu Tyr Phe Ile Asn Gln Gly Leu Phe Glu
  290 295 300
Leu Leu Phe Phe Trp Asn Thr Ser Leu Ser His Ala Gln Gln Tyr Arg
305 310 315 320
Trp Tyr Gln Met Leu Tyr Gln Ala Gly Val Phe Ala Ser Arg Ser Ser
                    325 330 335
Leu Arg Cys Cys Arg Ile Arg Phe Thr Trp Ala Leu Ala Leu Leu Gln
              340 345 350
Cys Leu Asn Leu Val Phe Leu Leu Ala Asp Val Trp Phe Gly Phe Leu
        355 360 365
Pro Ser Ile Tyr Leu Val Phe Leu Ile Ile Leu Tyr Glu Gly Leu Leu
    370 375 380
Gly Gly Ala Ala Tyr Val Asn Thr Phe His Asn Ile Ala Leu Glu Thr
385 390 395 400
Ser Asp Glu His Arg Glu Phe Ala Met Ala Ala Thr Cys Ile Ser Asp
                    405 410 415
Thr Leu Gly Ile Ser Leu Ser Gly Leu Leu Ala Leu Pro Leu His Asp
              420 425 430
Phe Leu Cys Gln Leu Ser
        435
CLN6 nucleotide sequence
(SEQ ID NO: 3)
atggaggcga cgcggaggcg gcagcacctg ggagcgacgg gcggcccagg cgcgcagctg 60
ggcgcctcct tcctgcaggc caggcatggc tctgtgagcg ctgatgaggc tgcccgcacg 120
gctcccttcc acctcgacct ctggttctac ttcacactgc agaactgggt tctggacttt 180
gggcgtccca ttgccatgct ggtattccct ctcgagtggt ttccactcaa caagcccagt 240
gttggggact acttccacat ggcctacaac gtcatcacgc cctttctctt gctcaagctc 300
atcgagcggt ccccccgcac cctgccacgc tccatcacgt acgtgagcat catcatcttc 360
atcatgggtg ccagcatcca cctggtgggt gactctgtca accaccgcct gctcttcagt 420
ggctaccagc accacctgtc tgtccgtgag aaccccatca tcaagaatct caagccggag 480
acgctgatcg actcctttga gctgctctac tattatgatg agtacctggg tcactgcatg 540
tggtacatcc ccttcttcct catcctcttc atgtacttca gcggctgctt tactgcctct 600
aaagctgaga gcttgattcc agggcctgcc ctgctcctgg tggcacccag tggcctgtac 660
tactggtacc tggtcaccga gggccagatc ttcatcctct tcatcttcac cttcttcgcc 720
atgctggccc tcgtcctgca ccagaagcgc aagcgcctct tcctggacag caacggcctc 780
ttcctcttct cctccttcgc actgaccctc ttgcttgtgg cgctctgggt cgcctggctg 840
tggaatgacc ctgttctcag gaagaagtac ccgggtgtca tctacgtccc tgagccctgg 900
gctttctaca cccttcacgt cagcagtcgg cactga 936
CLN6 amino acid sequence:
(SEQ ID NO: 4)
Met Glu Ala Thr Arg Arg Arg Gln His Leu Gly Ala Thr Gly Gly Pro
1 5 10 15
Gly Ala Gln Leu Gly Ala Ser Phe Leu Gln Ala Arg His Gly Ser Val
              20 25 30
Ser Ala Asp Glu Ala Ala Arg Thr Ala Pro Phe His Leu Asp Leu Trp
        35 40 45
Phe Tyr Phe Thr Leu Gln Asn Trp Val Leu Asp Phe Gly Arg Pro Ile
    50 55 60
Ala Met Leu Val Phe Pro Leu Glu Trp Phe Pro Leu Asn Lys Pro Ser
65 70 75 80
Val Gly Asp Tyr Phe His Met Ala Tyr Asn Val Ile Thr Pro Phe Leu
                85 90 95
Leu Leu Lys Leu Ile Glu Arg Ser Pro Arg Thr Leu Pro Arg Ser Ile
              100 105 110
Thr Tyr Val Ser Ile Ile Ile Phe Ile Met Gly Ala Ser Ile His Leu
          115 120 125
Val Gly Asp Ser Val Asn His Arg Leu Leu Phe Ser Gly Tyr Gln His
    130 135 140
His Leu Ser Val Arg Glu Asn Pro Ile Ile Lys Asn Leu Lys Pro Glu
145 150 155 160
Thr Leu Ile Asp Ser Phe Glu Leu Leu Tyr Tyr Tyr Asp Glu Tyr Leu
                    165 170 175
Gly His Cys Met Trp Tyr Ile Pro Phe Phe Leu Ile Leu Phe Met Tyr
              180 185 190
Phe Ser Gly Cys Phe Thr Ala Ser Lys Ala Glu Ser Leu Ile Pro Gly
        195 200 205
Pro Ala Leu Leu Leu Val Ala Pro Ser Gly Leu Tyr Tyr Trp Tyr Leu
    210 215 220
Val Thr Glu Gly Gln Ile Phe Ile Leu Phe Ile Phe Thr Phe Phe Ala
225 230 235 240
Met Leu Ala Leu Val Leu His Gln Lys Arg Lys Arg Leu Phe Leu Asp
                  245 250 255
Ser Asn Gly Leu Phe Leu Phe Ser Ser Phe Ala Leu Thr Leu Leu Leu
            260 265 270
Val Ala Leu Trp Val Ala Trp Leu Trp Asn Asp Pro Val Leu Arg Lys
      275 280 285
Lys Tyr Pro Gly Val Ile Tyr Val Pro Glu Pro Trp Ala Phe Tyr Thr
  290 295 300
Leu His Val Ser Ser Arg His
305 310
CLN8 nucleotide sequence
(SEQ ID NO: 5)
atgaatcctg cgagcgatgg gggcacatca gagagcattt ttgacctgga ctatgcatcc 60
tgggggatcc gctccacgct gatggtcgct ggctttgtct tctacttggg cgtctttgtg 120
gtctgccacc agctgtcctc ttccctgaat gccacttacc gttctttggt ggccagagag 180
aaggtcttct gggacctggc ggccacgcgt gcagtetttg gtgttcagag cacagccgca 240
ggcctgtggg ctctgctggg ggaccctgtg ctgcatgccg acaaggcgcg tggccagcag 300
aactggtgct ggtttcacat cacgacagca acgggattct tttgctttga aaatgttgca 360
gtccacctgt ccaacttgat cttccggaca tttgacttgt ttctggttat ccaccatctc 420
tttgcctttc ttgggtttct tggctgcttg gtcaatctcc aagctggcca ctatctagct 480
atgaccacgt tgctcctgga gatgagcacg ccctttacct gcgtttcctg gatgctctta 540
aaggcgggct ggtccgagtc tctgttttgg aagctcaacc agtggctgat gattcacatg 600
tttcactgcc gcatggttct aacctaccac atgtggtggg tgtgtttctg gcactgggac 660
ggcctggtca gcagcctgta tctgcctcat ttgacactgt tccttgtcgg actggctctg 720
cttacgctaa tcattaatcc atattggacc cataagaaga ctcagcagct tctcaatccg 780
gtggactgga acttcgcaca gccagaagcc aagagcaggc cagaaggcaa cgggcagctg 840
ctgcggaaga agaggccata g 861
CLN8 amino acid sequence
(SEQ ID NO: 6)
Met Asn Pro Ala Ser Asp Gly Gly Thr Ser Glu Ser Ile Phe Asp Leu
1 5 10 15
Asp Tyr Ala Ser Trp Gly Ile Arg Ser Thr Leu Met Val Ala Gly Phe
              20 25 30
Val Phe Tyr Leu Gly Val Phe Val Val Cys His Gln Leu Ser Ser Ser
        35 40 45
Leu Asn Ala Thr Tyr Arg Ser Leu Val Ala Arg Glu Lys Val Phe Trp
    50 55 60
Asp Leu Ala Ala Thr Arg Ala Val Phe Gly Val Gln Ser Thr Ala Ala
65 70 75 80
Gly Leu Trp Ala Leu Leu Gly Asp Pro Val Leu His Ala Asp Lys Ala
                  85 90 95
Arg Gly Gln Gln Asn Trp Cys Trp Phe His Ile Thr Thr Ala Thr Gly
            100 105 110
Phe Phe Cys Phe Glu Asn Val Ala Val His Leu Ser Asn Leu Be Phe
        115 120 125
Arg Thr Phe Asp Leu Phe Leu Val Ile His His Leu Phe Ala Phe Leu
    130 135 140
Gly Phe Leu Gly Cys Leu Val Asn Leu Gln Ala Gly His Tyr Leu Ala
145 150 155 160
Met Thr Thr Leu Leu Leu Glu Met Ser Thr Pro Phe Thr Cys Val Ser
                  165 170 175
Trp Met Leu Leu Lys Ala Gly Trp Ser Glu Ser Leu Phe Trp Lys Leu
            180 185 190
Asn Gln Trp Leu Met Ile His Met Phe His Cys Arg Met Val Leu Thr
        195 200 205
Tyr His Met Trp Trp Val Cys Phe Trp His Trp Asp Gly Leu Val Ser
    210 215 220
Ser Leu Tyr Leu Pro His Leu Thr Leu Phe Leu Val Gly Leu Ala Leu
225 230 235 240
Leu Thr Leu Ile Ile Asn Pro Tyr Trp Thr His Lys Lys Thr Gln Gln
                  245 250 255
Leu Leu Asn Pro Val Asp Trp Asn Phe Ala Gln Pro Glu Ala Lys Ser
            260 265 270
Arg Pro Glu Gly Asn Gly Gln Leu Leu Arg Lys Lys Arg Pro
      275 280 285
CB promoter
(SEQ ID NO: 7)
ccacgttctg cttcactctc cccatctccc ccccctcccc acccccaatt ttgtatttat 60
ttatttttta attattttgt gcagcgatgg gggcgggggg gggggggggg cgcgcgccag 120
gcggggcggg gcggggcgag gggcggggcg gggcgaggcg gagaggtgcg gcggcagcca 180
atcagagcgg cgcgctccga aagtttcctt ttatggcgag gcggcggcgg cggcggccct 240
ataaaaagcg aagcgcgcgg cgggcgggag 270
CMV promoter
(SEQ ID NO: 8)
cgttacataa cttacggtaa atggcccgcc tggctgaccg cccaacgacc cccgcccatt 60
gacgtcaata atgacgtatg ttcccatagt aacgccaata gggactttcc attgacgtca 120
atgggtggag tatttacggt aaactgccca cttggcagta catcaagtgt atcatatgcc 180
aagtacgccc cctattgacg tcaatgacgg taaatggccc gcctggcatt atgcccagta 240
catgacctta tgggactttc ctacttggca gtacatctac 280
P546 Promoter
(SEQ ID NO: 9)
gaacaacgccaggctcctcaacaggcaactttgctacttctacagaaaatgataataaag
aaatgctggtgaagtcaaatgcttatcacaatggtgaactactcagcagggaggctctaa
taggcgccaagagcctagacttccttaagcgccagagtccacaagggcccagttaatcct
caacattcaaatgctgcccacaaaaccagcccctctgtgccctagccgcctcttttttcc
aagtgacagtagaactccaccaatccgcagctgaatggggtccgcctcttttccctgcct
aaacagacaggaactcctgccaattgagggcgtcaccgctaaggctccgccccagcctgg
gctccacaaccaatgaagggtaatctcgacaaagagcaaggggtggggcgcgggcgcgca
ggtgcagcagcacacaggctggtcgggagggcggggcgcgacgtctgccgtgcggggtcc
cggcatcggttgcgcgcgcgctccctcctctcggagagagggctgtggtaaaacccgtcc
ggaaaa
```

What is claimed:

1. A composition comprising a gene therapy vector and a glycoside hydrolase enzyme.

2. The composition of claim 1, wherein the glycoside hydrolase enzyme is neuraminidase, lactase, amylase, chitinase, cellulase, sucrase, maltase, invertase, or lysozyme.

3. The composition of claim 1 or 2, wherein the gene therapy vector is AAV8, AA9 or Anc80.

4. The composition of any one of claims 1-3, wherein the composition is formulated for local intravenous delivery, sub-retinal delivery, intravitreal delivery or intrathecal delivery.

5. The composition of any one of claims 1-4, wherein the gene therapy vector and the glycoside hydrolase enzyme are admixed for administration simultaneously.

6. A kit comprising for delivering a transgene to a retinal cell of a subject comprising a a gene therapy vector and a glycoside hydrolase enzyme.

7. The kit of claim 6, wherein the the glycoside hydrolase enzyme is neuraminidase, lactase, amylase, chitinase, cellulase, sucrase, maltase, invertase, or lysozyme.

8. The kit of claim 6 or 7, wherein the gene therapy vector is AAV8, AA9 or Anc80.

9. A method of delivering a transgene to a retinal cell in a subject comprising administering i) a gene therapy vector encoding the transgene, and ii) a glycoside hydrolase enzyme to the subject.

10. A method of treating visual impairment or a vision-related disorder in a subject comprising administering i) a gene therapy vector encoding the transgene, and ii) a glycoside hydrolase enzyme to the subject, wherein delivery of the transgene effectively treats the visual impairment or the vision-related disorder.

11. The method of claim 9 or 10, wherein the glycoside hydrolase enzyme is neuraminidase, lactase, amylase, chitinase, cellulase, sucrase, maltase, invertase, or lysozyme.

12. The method of any one of claims 9-11, wherein the retinal cell is a bipolar cell, rod photoreceptor cell, cone photoreceptor cell, ganglion cell, Mueller glia cell, microglia cell, horizontal cell or amacrine cell.

13. The method of any one of claims 9-12, wherein the gene therapy vector is administered to the subject using local intravenous delivery, sub-retinal delivery, intravitreous delivery or intrathecal delivery.

14. The method of any one of claims 9-13, wherein the gene therapy vector encoding the transgene and the glycoside hydrolase enzyme are administered simultaneously to the subject.

15. The method of claim 14, wherein the gene therapy vector and the glycoside hydrolase enzyme are admixed.

16. The method of any one of claims 9-14, wherein the gene therapy vector and the glycoside hydrolase enzyme are administered separately.

17. The method of any one of claims 10-16 wherein the vision-related disorder is Batten disease, congenital cataracts, congenital glaucoma, retinal degeneration, optic atrophy, eye malformations. Strabismus, ocular misalignment, glaucoma, wet age-related macular degeneration, dry age-related macular degeneration, retinitis pigmentosa, choroideremia, Leber congenital amaurosis, Leber's hereditary optic neuropathy, early onset retinal dystrophy, achromatopsia, x-linked retinoschisis, Usher Syndrome 1B, neovascular age-related macular degeneration, Stargardt's macular degeneration, diabetic macular degeneration, or diabetic macular edema.

18. A composition for delivering a transgene to a retinal cell in a subject, wherein the composition comprises i) a gene therapy vector encoding the transgene, and ii) a glycoside hydrolase enzyme.

19. A composition for treating visual impairment or a vision-related disorder in a subject, wherein the composition comprises i) a gene therapy vector encoding the transgene, and ii) a glycoside hydrolase enzyme.

20. A composition for delivering a transgene to a retinal cell in a subject, wherein the composition comprises a gene therapy vector encoding the transgene, wherein the composition is administered with a second composition comprising a glycoside hydrolase enzyme.

21. A composition for treating a treating visual impairment or a vision-related disorder in a subject, wherein the composition comprises a gene therapy vector encoding the transgene, wherein the composition is administered with a second composition comprising a glycoside hydrolase enzyme.

22. A composition for delivering a transgene to a retinal cell in a subject, wherein the composition comprises a glycoside hydrolase enzyme, wherein the composition is administered with a second composition comprising a gene therapy vector encoding the transgene.

23. A composition for treating a treating visual impairment or a vision-related disorder in a subject, wherein the composition comprises a glycoside hydrolase enzyme, wherein the composition is administered with a second composition comprising a gene therapy vector encoding the transgene.

24. The composition of any one of claims 19-23, wherein the glycoside hydrolase enzyme is neuraminidase, lactase, amylase, chitinase, cellulase, sucrase, maltase, invertase, or lysozyme.

25. The composition of any one of claims 19-24, wherein the retinal cell is a bipolar cell, rod photoreceptor cell, cone photoreceptor cell, ganglion cell, Mueller glia cell, microglia cell, horizontal cell or amacrine cell.

26. The composition of any one of claims 19-25, wherein the composition is formulated for local intravenous delivery, sub-retinal delivery, intravitreal delivery or intrathecal delivery.

27. The composition of any one of claims 20-26, wherein the composition and the second composition are administered simultaneously to the subject.

28. The composition of any one of claims 20-27, wherein the composition and the second composition are admixed.

29. The composition of any one of claims 20-27, wherein the composition and the second composition are administered separately.

30. The composition of any one of claim 19, 21 or 23-29, wherein the vision-related disorder is Batten disease, congenital cataracts, congenital glaucoma, retinal degeneration, optic atrophy, eye malformations. Strabismus, ocular misalignment, glaucoma, wet age-related macular degeneration, dry age-related macular degeneration, retinitis pigmentosa, choroideremia, Leber congenital amaurosis, Leber's hereditary optic neuropathy, early onset retinal dystrophy, achromatopsia, x-linked retinoschisis, Usher Syndrome 1B, neovascular age-related macular degeneration, Stargardt's macular degeneration, diabetic macular degeneration, or diabetic macular edema.

31. Use of composition for the preparation of a medicament for delivering a transgene to a retinal cell in a subject, wherein the composition comprises i) a gene therapy vector encoding the transgene, and ii) a glycoside hydrolase enzyme.

32. Use of a composition for the preparation of a medicament for treating visual impairment or a vision-related disorder in a subject, wherein the composition comprises i) a gene therapy vector encoding the transgene, and ii) a glycoside hydrolase enzyme.

33. Use of a gene therapy vector encoding a transgene for the preparation of a medicament for delivering a transgene to a retinal cell in a subject, wherein the medicament is administered with a composition comprising a glycoside hydrolase enzyme.

34. Use of a gene therapy vector encoding a transgene for the preparation of a medicament for treating visual impairment or a vision-related disorder in a subject, wherein the medicament is administered with a composition comprising a glycoside hydrolase enzyme.

35. Use of glycoside hydrolase enzyme for the preparation of a medicament for delivering a transgene to a retinal cell in a subject, wherein the medicament is administered with a composition comprising a gene therapy vector encoding the transgene.

36. Use of glycoside hydrolase enzyme for the preparation of a medicament for treating a treating visual impairment or a vision-related disorder in a subject, wherein the medicament is administered with a composition comprising a gene therapy vector encoding the transgene.

37. The use of any one of claims 32-36, wherein the glycoside hydrolase enzyme is neuraminidase, lactase, amylase, chitinase, cellulase, sucrase, maltase, invertase, or lysozyme.

38. The use of any one of claims 32-37, wherein the retinal cell is a bipolar cell, rod photoreceptor cell, cone photoreceptor cell, ganglion cell, Mueller glia cell, microglia cell, horizontal cell or amacrine cell.

39. The use of any one of claims 32-38, wherein the medicament is formulated for local intravenous delivery, sub-retinal delivery, intravitreal delivery or intrathecal delivery.

40. The use of any one of claims 33-39, wherein the medicament and the composition are administered simultaneously to the subject.

41. The use of any one of claims 33-39, wherein the composition and the composition are admixed.

42. The use of any one of claims 33-39, wherein the medicament and the composition are administered separately.

43. The use of any one of claim 32, 34 or 36-42, wherein the vision-related disorder is Batten disease, congenital cataracts, congenital glaucoma, retinal degeneration, optic atrophy, eye malformations. Strabismus, ocular misalignment, glaucoma, wet age-related macular degeneration, dry age-related macular degeneration, retinitis pigmentosa, choroideremia, Leber congenital amaurosis, Leber's hereditary optic neuropathy, early onset retinal dystrophy, achromatopsia, x-linked retinoschisis, Usher Syndrome 1B, neovascular age-related macular degeneration, Stargardt's macular degeneration, diabetic macular degeneration, or diabetic macular edema.


SEQUENCE LISTING (9 sequences)

SEQ ID NO: 1 - DNA, 1317 bp, Homo sapiens
atgggaggctgtgcaggctcgcggcggcgcttttcggattccgagggggaggagaccgtcccggagccccggctccctctgttggaccatcagggcgcgcattggaagaacgcggtgggcttctggctgctgggcctttgcaacaacttctcttatgtggtgatgctgagtgccgcccacgacatccttagccacaagaggacatcgggaaaccagagccatgtggacccaggcccaacgccgatcccccacaacagctcatcacgatttgactgcaactctgtctctacggctgctgtgctcctggcggacatcctccccacactcgtcatcaaattgttggctcctcttggccttcacctgctgccctacagcccccgggttctcgtcagtgggatttgtgctgctggaagcttcgtcctggttgccttttctcattctgtggggaccagcctgtgtggtgtggtcttcgctagcatctcatcaggccttggggaggtcaccttcctctccctcactgccttctaccccagggccgtgatctcctggtggtcctcagggactgggggagctgggctgctgggggccctgtcctacctgggcctcacccaggccggcctctcccctcagcagaccctgctgtccatgctgggtatccctgccctgctgctggccagctatttcttgttgctcacatctcctgaggcccaggaccctggaggggaagaagaagcagagagcgcagcccggcagcccctcataagaaccgaggccccggagtcgaagccaggctccagctccagcctctcccttcgggaaaggtggacagtgttcaagggtctgctgtggtacattgttcccttggtcgtagtttactttgccgagtatttcattaaccagggactttttgaactcctctttttctggaacacttccctgagtcacgctcagcaataccgctggtaccagatgctgtaccaggctggcgtctttgcctcccgctcttctctccgctgctgtcgcatccgtttcacctgggccctggccctgctgcagtgcctcaacctggtgttcctgctggcagacgtgtggttcggctttctgccaagcatctacctcgtcttcctgatcattctgtatgaggggctcctgggaggcgcagcctacgtgaacaccttccacaacatcgccctggagaccagtgatgagcaccgggagtttgcaatggcggccacctgcatctctgacacactggggatctccctgtcggggctcctggctttgcctctgcatgacttcctctgccagctctcctga

SEQ ID NO: 2 - protein, 438 aa, Homo sapiens
MGGCAGSRRRFSDSEGEETVPEPRLPLLDHQGAHWKNAVGFWLLGLCNNFSYVVMLSAAHDILSHKRTSGNQSHVDPGPTPIPHNSSSRFDCNSVSTAAVLLADILPTLVIKLLAPLGLHLLPYSPRVLVSGICAAGSFVLVAFSHSVGTSLCGVVFASISSGLGEVTFLSLTAFYPRAVISWWSSGTGGAGLLGALSYLGLTQAGLSPQQTLLSMLGIPALLLASYFLLLTSPEAQDPGGEEEAESAARQPLIRTEAPESKPGSSSSLSLRERWTVFKGLLWYIVPLVVVYFAEYFINQGLFELLFFWNTSLSHAQQYRWYQMLYQAGVFASRSSLRCCRIRFTWALALLQCLNLVFLLADVWFGFLPSIYLVFLIILYEGLLGGAAYVNTFHNIALETSDEHREFAMAATCISDTLGISLSGLLALPLHDFLCQLS

SEQ ID NO: 3 - DNA, 936 bp, Homo sapiens
atggaggcgacgcggaggcggcagcacctgggagcgacgggcggcccaggcgcgcagctgggcgcctccttcctgcaggccaggcatggctctgtgagcgctgatgaggctgcccgcacggctcccttccacctcgacctctggttctacttcacactgcagaactgggttctggactttgggcgtcccattgccatgctggtattccctctcgagtggtttccactcaacaagcccagtgttggggactacttccacatggcctacaacgtcatcacgccctttctcttgctcaagctcatcgagcggtccccccgcaccctgccacgctccatcacgtacgtgagcatcatcatcttcatcatgggtgccagcatccacctggtgggtgactctgtcaaccaccgcctgctcttcagtggctaccagcaccacctgtctgtccgtgagaaccccatcatcaagaatctcaagccggagacgctgatcgactcctttgagctgctctactattatgatgagtacctgggtcactgcatgtggtacatccccttcttcctcatcctcttcatgtacttcagcggctgctttactgcctctaaagctgagagcttgattccagggcctgccctgctcctggtggcacccagtggcctgtactactggtacctggtcaccgagggccagatcttcatcctcttcatcttcaccttcttcgccatgctggccctcgtcctgcaccagaagcgcaagcgcctcttcctggacagcaacggcctcttcctcttctcctccttcgcactgaccctcttgcttgtggcgctctgggtcgcctggctgtggaatgaccctgttctcaggaagaagtacccgggtgtcatctacgtccctgagccctgggctttctacacccttcacgtcagcagtcggcactga

SEQ ID NO: 4 - protein, 311 aa, Homo sapiens
MEATRRRQHLGATGGPGAQLGASFLQARHGSVSADEAARTAPFHLDLWFYFTLQNWVLDFGRPIAMLVFPLEWFPLNKPSVGDYFHMAYNVITPFLLLKLIERSPRTLPRSITYVSIIIFIMGASIHLVGDSVNHRLLFSGYQHHLSVRENPIIKNLKPETLIDSFELLYYYDEYLGHCMWYIPFFLILFMYFSGCFTASKAESLIPGPALLLVAPSGLYYWYLVTEGQIFILFIFTFFAMLALVLHQKRKRLFLDSNGLFLFSSFALTLLLVALWVAWLWNDPVLRKKYPGVIYVPEPWAFYTLHVSSRH

SEQ ID NO: 5 - DNA, 861 bp, Homo sapiens
atgaatcctgcgagcgatgggggcacatcagagagcatttttgacctggactatgcatcctgggggatccgctccacgctgatggtcgctggctttgtcttctacttgggcgtctttgtggtctgccaccagctgtcctcttccctgaatgccacttaccgttctttggtggccagagagaaggtcttctgggacctggcggccacgcgtgcagtctttggtgttcagagcacagccgcaggcctgtgggctctgctgggggaccctgtgctgcatgccgacaaggcgcgtggccagcagaactggtgctggtttcacatcacgacagcaacgggattcttttgctttgaaaatgttgcagtccacctgtccaacttgatcttccggacatttgacttgtttctggttatccaccatctctttgcctttcttgggtttcttggctgcttggtcaatctccaagctggccactatctagctatgaccacgttgctcctggagatgagcacgccctttacctgcgtttcctggatgctcttaaaggcgggctggtccgagtctctgttttggaagctcaaccagtggctgatgattcacatgtttcactgccgcatggttctaacctaccacatgtggtgggtgtgtttctggcactgggacggcctggtcagcagcctgtatctgcctcatttgacactgttccttgtcggactggctctgcttacgctaatcattaatccatattggacccataagaagactcagcagcttctcaatccggtggactggaacttcgcacagccagaagccaagagcaggccagaaggcaacgggcagctgctgcggaagaagaggccatag

SEQ ID NO: 6 - protein, 286 aa, Homo sapiens
MNPASDGGTSESIFDLDYASWGIRSTLMVAGFVFYLGVFVVCHQLSSSLNATYRSLVAREKVFWDLAATRAVFGVQSTAAGLWALLGDPVLHADKARGQQNWCWFHITTATGFFCFENVAVHLSNLIFRTFDLFLVIHHLFAFLGFLGCLVNLQAGHYLAMTTLLLEMSTPFTCVSWMLLKAGWSESLFWKLNQWLMIHMFHCRMVLTYHMWWVCFWHWDGLVSSLYLPHLTLFLVGLALLTLIINPYWTHKKTQQLLNPVDWNFAQPEAKSRPEGNGQLLRKKRP

SEQ ID NO: 7 - DNA, 270 bp, Homo sapiens
ccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggag

SEQ ID NO: 8 - DNA, 280 bp, Homo sapiens
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctac

SEQ ID NO: 9 - DNA, 546 bp, Homo sapiens
gaacaacgccaggctcctcaacaggcaactttgctacttctacagaaaatgataataaagaaatgctggtgaagtcaaatgcttatcacaatggtgaactactcagcagggaggctctaataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaaa